(19) United States
(12) Patent Application Publication
(10) Pub. No.: US 2020/0191155 A1
(43) Pub. Date: Jun. 18, 2020

(54) ODOR DELIVERY DEVICE

(71) Applicant: ScentScore Inc., New Milford, CT (US)

(72) Inventors: Stuart Jay Firestein, New Milford, CT (US); Christophe Laudamiel, New York, NY (US); Dmitry Rinberg, New York, NY (US); Tatyana Tabachnik, New York, NY (US)

(73) Assignee: ScentScore Inc., New Milford, CT (US)

(21) Appl. No.: 16/718,555

(22) Filed: Dec. 18, 2019

Related U.S. Application Data
(60) Provisional application No. 62/781,052, filed on Dec. 18, 2018.

(51) Int. Cl. F04D 27/00; F04D 25/16; F04D 29/00; G05B 19/042; A61L 9/12; A63F 13/25

(52) U.S. Cl. CPC F04D 27/00; F04D 25/16; F04D 29/002; G05B 19/042; A61L 9/125; A63F 2300/8082; G05B 2219/25257; A61L 2209/134; A61L 2209/133; A61L 2209/111; A63F 13/25

(57) ABSTRACT

In certain embodiments, an odor delivery device for a VR/AR system has independently controllable micropumps and dedicated tubing for each micropump that dispenses pumped air from the device. The tubings can receive scented material such that, when air is pumped by the corresponding micropump through a tubing, scented air flow is dispensed by the tubing from the device. When multiple tubings have multiple differently scented materials, corresponding differently scented air flows concurrently dispensed by the tubings are mixed together only outside the device. In one implementation, a pump unit has the micropumps and tubing for each micropump and a cartridge unit having tubing for each micropump in the pump unit, can be removably mated to the pump unit. When the cartridge unit is mated to the pump unit, each tubing in the cartridge unit mates with corresponding tubing in the pump unit.

### CROSS-REFERENCE TO RELATED APPLICATIONS

This application claims the benefit of the filing date of U.S. provisional application No. 62/781,052, filed on Dec. 19, 2018 as attorney docket no. 1364.001PROV, the teachings of which are incorporated herein by reference in their entirety.

### BACKGROUND

### Field of the Invention

The present invention relates to odor delivery systems and, more particularly but not exclusively, to odor delivery systems for virtual reality (VR) and augmented reality (AR) applications.

### Description of the Related Art

This section introduces aspects that may help facilitate a better understanding of the invention. Accordingly, the statements of this section are to be read in this light and are not to be understood as admissions about what is prior art or what is not prior art.

Systems for delivering odors, scents, and fragrances to users exist, but have limitations and disadvantages that are addressed by the embodiments of this disclosure.

### SUMMARY

Problems in the prior art are addressed in accordance with the principles of the present invention.

In one embodiment, the present invention is an odor delivery system as described below.

### BRIEF DESCRIPTION OF THE DRAWINGS

Embodiments of the invention will become more fully apparent from the following detailed description, the appended claims, and the accompanying drawings in which like reference numerals identify similar or identical elements.

FIG. 1 is a schematic diagram of an odor delivery system according to one embodiment;

FIG. 2A is a cross-sectional side view of the odor delivery device of FIG. 1;

FIG. 2B is a plan view of the proximal end of the micropump housing of FIG. 2A;

FIG. 2C is a perspective view of a micropump of FIG. 2A;

FIG. 3 is a cross-sectional side view of the odor cartridge and the nose piece of FIG. 2A;

FIG. 4A is a perspective view of the odor delivery device of FIG. 1;

FIG. 4B is a cross-sectional perspective view of the housing unit of FIG. 2A;

FIG. 5 is a block diagram of the electronic control unit of FIG. 1;

FIG. 6A is a schematic of a configuration for measuring airflow from the odor delivery device of FIG. 1 using an airflow detector;

FIG. 6B is a schematic of a configuration for measuring concentration temporal profiles of odor delivery for the odor delivery device of FIG. 1 using a photo-ionization detector;

FIGS. 7A1-7A3 are graphical representations of control pulse temporal profiles;

FIGS. 7B1-7B3 are graphical representations of the concentration of different odorants as a function of time for the control pulses of FIGS. 7A1-7A3;

FIG. 8A is graphical representations of the control pulse temporal profiles for different pulse amplitudes;

FIG. 8B are graphs of the concentration of an odorant for the different control pulse amplitudes of FIG. 8A;

FIG. 9 is a schematic diagram of air flow during user inhalation;

FIG. 10 is a schematic diagram of a configuration of the odor delivery device and the electronic control unit of FIG. 1 with a virtual reality device;

FIG. 11 is a schematic diagram of a possible positioning of an odor delivery device on a user's head together with a virtual reality device;

FIG. 12 is a schematic diagram of an odor delivery device having two micropump blocks, two odor cartridges, and one nose piece;

FIG. 13A is a schematic diagram of an odor delivery system that can be synchronized with a user's respiratory pattern; and

FIG. 13B is a timing diagram showing the synchronization of odor delivery with the user's respiratory pattern.

### DETAILED DESCRIPTION

Detailed illustrative embodiments of the present disclosure are disclosed herein. However, specific structural and functional details disclosed herein are merely representative for purposes of describing example embodiments of the present disclosure. The present disclosure may be embodied in many alternate forms and should not be construed as limited to only the embodiments set forth herein. Further, the terminology used herein is for the purpose of describing particular embodiments only and is not intended to be limiting of example embodiments of the disclosure.

As used herein, the singular forms “a,” “an,” and “the,” are intended to include the plural forms as well, unless the context clearly indicates otherwise. It further will be understood that the terms “comprises,” “comprising,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

This disclosure describes individual/personal odor delivery systems having an odor delivery device that will deliver small quantities of odorants in a precise and controlled manner in space, time, character, and quantity. Unlike previous iterations of odor delivery devices, this device is small, wearable, and affects only the immediate personal space of the user. The device can be utilized to supply a scent track coordinated with movie, game, on-line, virtual, or augmented reality scripts. The device can also be utilized for personal advertising, education, medical devices, and demonstration purposes.

The device is lightweight and incorporates new odor delivery mechanisms as well new methods for mixing and storing odors.

We present here a device for delivering odorant compounds in extremely small quantities limited to a region directly below the nostrils, thereby allowing for a large array of scent interpretations and characters, with very little to no contamination.

A “scent track” of scents is perceivable only to the directed individual with no spreading of odors into the general environment. The terms “odor,” “scent”, and “fragrance” are used synonymously herein to refer both to a substance that can produce a perception of a particular smell by the user of an odor delivery system as well as to the perception itself, while the term “odorant” is limited to a substance that can produce such a perception.

Implementations of the device include one or more of the following advantages:
Odor delivery is extremely precise in space (i.e., a small volume under the nostrils of the user), in time (i.e., time on, duration, time off), in strength (i.e., intensity), and with fast switching between odors allowing precise coordination with an audio/video/touch/motion stimulus (e.g., music, movie, walking, etc.).
The device is wearable or can be used as a standalone and is designed for personal individual use. The device is lightweight and self-contained, and can be integrated with other systems, such as movies, audio, video, VR (virtual reality), and AR (augmented reality).
The device preserves the freshness of the scents and allows close and safe personal contact (e.g., wearable on the head or other sensitive body areas).
The device imposes no known limitations on odor choice. A wide range of odor creations are possible. There are no limitations for high volatility or flammability or for low vapor point (solids/resins).
The device is tunable; i.e., the device can deliver odors in adjustable concentrations. The device allows presentation of mixtures of odors with variable ratios between individual components.
The device can deliver temporarily variable concentrations of single and multiple odors, like concentrations ramping up and down, thus creating a realistic experience for a user.
Because the device delivers only small amounts of odor, there is no detectable build-up of odors in a normal room or between individuals. That is, there is no contamination of a space beyond the head of the individual using the device.
The point of odor delivery is close to the user's nostril(s).
The air flow is very slow and almost non-detectable, thus creating a more-realistic scent experience. The flow is just sufficient to produce an odor perception without being disruptive.
The low air flow also prevents dryness in the nostrils or other discomforts.
The device has very little to non-detectable contamination between odor reservoirs.
The individual odor lines are fully separated and arrive to a common point only at the delivery point. That is, there are no mixing chambers or common paths for different odors.
The device generates negligible heat.
The device is quiet. There are no moving parts or fans, and no vibration in the range of human sensitivity, making the device wearable, appropriate for personal use, and acceptable in an enclosed audio-video environment.
The device incorporates an independent purge line or “blank.”
The device is programmable and can be controlled by a computer interface or smartphone app.
The device has the capacity for a large number of puffs per cartridge.
The device possesses or can possess a disposable tip to avoid cross-contamination between users.
Multiple devices can be combined together, thus allowing for a major increase of the number of odors.
The device allows for control of overall odor intensity by an individual user.

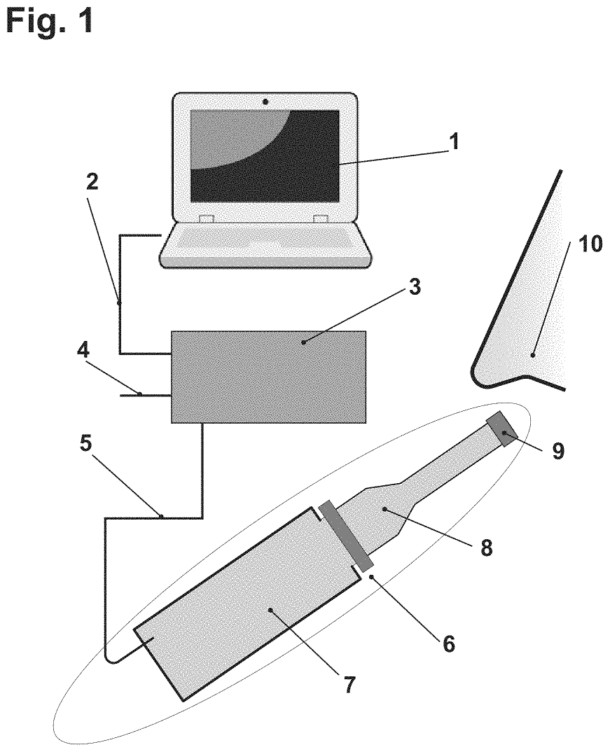

FIG. 1 shows one embodiment of an odor delivery system of the disclosure. The system is controlled by a processor-based computer 1, which is connected via a USB cable 2 to an electronic control unit 3. The computer 1 and the electronic control unit 3 may be said to form a controller for the system of FIG. 1. The electronic control unit 3 is powered by a 24 Vdc power line 4. The electronic control unit 3 is connected via a multi-wire cable 5 to the odor delivery device 6. The odor delivery device 6 includes a micropump unit 7, a replaceable odor cartridge 8, and an optional replaceable nose piece 9 (e.g., made of a suitable polymer) for use by an individual user. The odor delivery device 6 is positioned in such a way that the tip of the nose piece 9 is located in close proximity (e.g., 0 to 4 inches) to a user's nose 10. In FIG. 1, the micropump unit 7 abuts the odor cartridge unit 8, but does not abut in alternative embodiments. In FIG. 1, the cartridge unit 8 abuts the nose piece 9, but does not abut in alternative embodiments. In any case, the cartridge unit 8 should be in near proximity from the nose piece 9 in order to preserve odor freshness and ensemble.

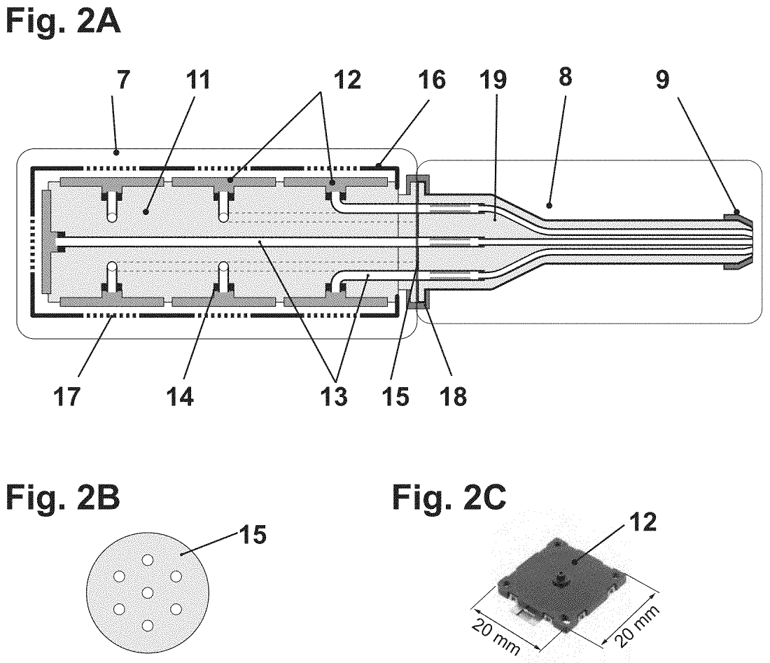

FIG. 2A is a cross-sectional side view of the odor delivery device 6 of FIG. 1. The odor delivery device 6 includes three main parts: the micropump unit 7, the odor cartridge 8, and the nose piece 9. The micropump unit 7 provides a rectilinear housing 11 for a bundle of seven micropumps 12. Six individual micropumps 12 are located on two opposite sides of the micropump housing 11, and the seventh micropump 12 is located on the bottom side of the micropump housing 11. The housing unit 11 has seven embedded tubings 13, each having an internal diameter of about 1/16 inch or smaller (e.g., between 2 mm and 0.5 mm). Each micropump 12 is connected to a corresponding tubing 13 via a gasket 14. The outlets of all seven tubings 13 are located at the top surface 15 of the housing 11. The top surface 15 of the housing unit 11 is shown on FIG. 2B.

Each micropump 12 is a piezo microblower, such as a Microblower MZB1001T02 from Murata Manufacturing Co. of Japan, which provides an air flow up to about 1 L/min if no load is attached. Each micropump 12 is driven by 24 kHz ac current with variable amplitude up to 20 V peak-to-peak. During operation, each micropump 12 does not emit any audible sound except from an airflow itself. A micropump 12 is shown on FIG. 2C. each micropump 12 is attached to the housing 11 with four micro-screws (not shown).

The housing 11 containing the micropumps 12 is covered by a protective shield 16 with seven ventilation grids 17 for air inlets to the seven individual micropumps 12. The housing 11 also has an inlet (not shown in FIG. 2A) for the multi-wire cable 5 of FIG. 2A to provide a separate electrical connection from the electronic control unit 3 of FIG. 2A to power each of the micropumps 12. In one possible implementation, the dimensions of the external protective shield 16 are 3 inch long×1.25 inch high×1.5 inch wide.

The odor cartridge 8 is attached to the micropump unit 7 with a clamp 18. An individual nose piece 9 is attached to the outlet of the odor cartridge 8 to insure each user's individual hygiene and prevent cross-contamination between users and between scent tracks.

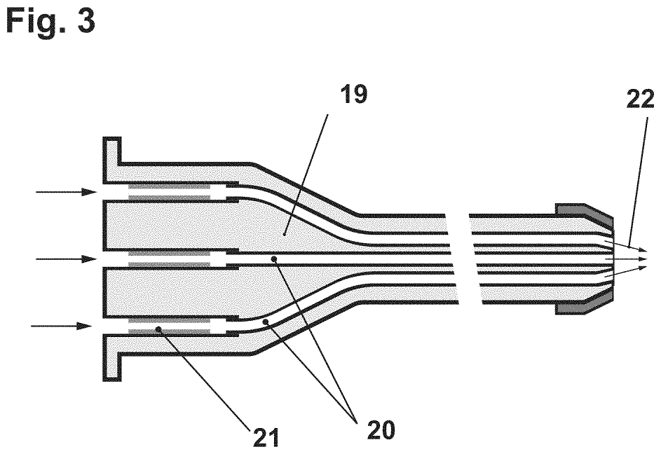

A scaled view of the odor cartridge 8 is shown in FIG. 3. The odor cartridge housing 19 encapsulates seven lines (tubing) 20 made of Teflon or other suitable material. Seven separate lines 20 are used to prevent cross-contamination between individual odorants. The inlet of each line 20 has an internal diameter of about 1/16 inch, and the outlet of each line 20 has a smaller diameter (e.g., about 1/32 inch) to provide an air resistance and reduce odor diffusion from the cartridge 8 to an outside space. In alternative implementations, the internal diameter of each line 20 is between about 1 mm and 0.5 mm. A piece of (e.g., polymer) sponge material 21 impregnated with a different, suitable odorant can be inserted into each of one or more lines 20 from the inlet side. In some implementations, for each line 20 having such impregnated sponge material 20, the impregnated sponge material 21 fills more than two-thirds of the volume of the line 20. In some implementations, the sponge material 20 absorbs less than half its weight in odorant. In some implementations, the sponge material 20 absorbs 25%, 50%, or even 100% more odorant than (e.g., polymer) material of the lines 20. The central portion of each such tubing 20 can be used as an odorant channel. Note that one or more lines 20 can be used for the delivery of clean air by not inserting an odorant-impregnated material 21 into those lines 20. The outlets 22 of all lines (tubing) 20 are tilted towards the central axis in order to direct a flow of odorized air to the same small area. The length of the odor cartridge 8 can be, but does not have to be, between 1 and 6 inches.

In the embodiment of FIG. 3, the outlets 22 of the tubings 20 extend all the way to the end of the odor cartridge 8, such that any mixing of air flows from different tubings 20 occurs outside of the odor delivery device 6. In alternative embodiments, the tubings 20 end a short distance from the end of the odor cartridge 8 such that there is a relatively small interior volume near the end of the odor cartridge 8 within which the air flows from different tubings 20 will mix before the resultant mixed air flows are dispensed from the end of the odor delivery device 6. In some implementations, the length of that interior volume is less than the sum of the inner diameters of at the ends of the tubings 20. In some implementations, the length of the interior volume is less than or equal to 6 mm. In some implementations, the length of the interior volume is less than or equal to 1 mm.

Due to the different lengths of tubing 13 in the housing 11 and of tubing 20 in the odor cartridge 8, the micropumps 12 may produce different air flows at the outlet of the nose piece 9. The software and electronic control of the micropump power implemented by the electronic control unit 3 can compensate for such flow differences to ensure similar airflows.

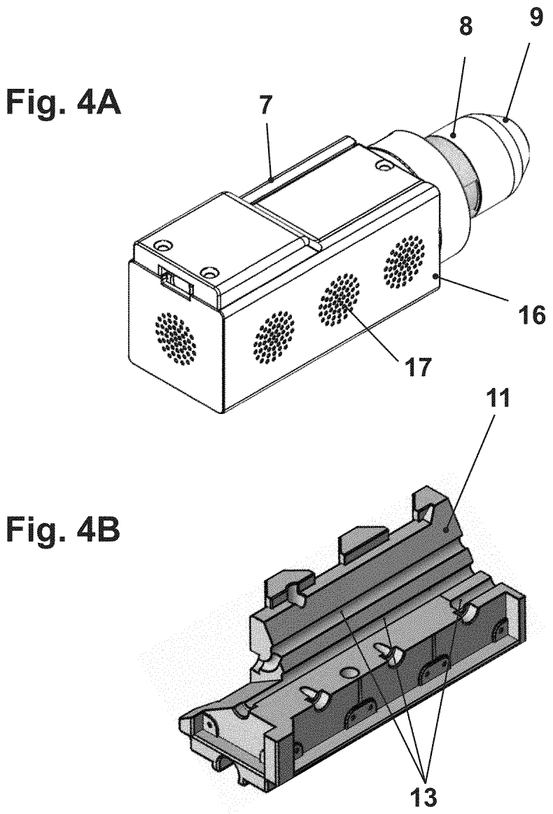

FIG. 4A shows a perspective view of the odor delivery device 6. FIG. 4B shows a cross-sectional perspective view of the housing unit 11 with embedded tubing 13. The design of the odor delivery device 6 was optimized for manufacturing using 3D printer technology, for easy mounting of the micropumps 12, and aerodynamics of the air flow channels, to ensure minimal air resistance from the micropump 12 to the outlet of the embedded tubing 13.

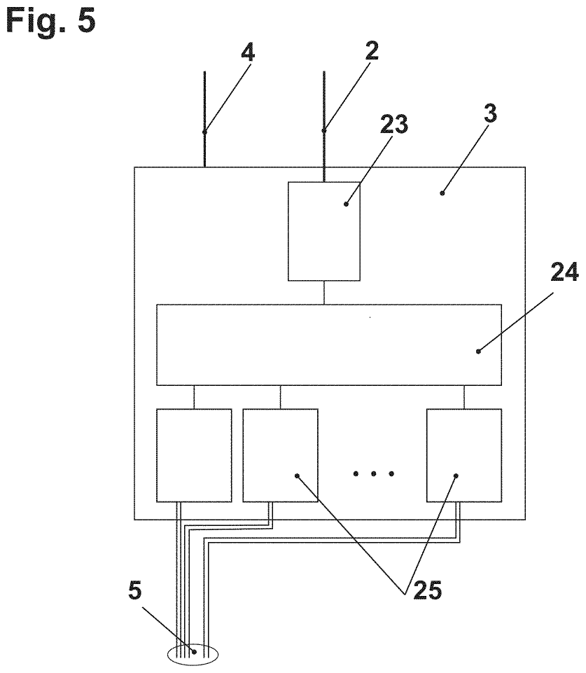

FIG. 5 shows a block diagram of the electronic control unit 3, which includes a microprocessor 23 (e.g., a Teensy microcontroller from PJRC.COM, LLC, of Sherwood, Oreg.), a distribution electronics board 24, and seven independent micropump drivers 25. The microprocessor 23 is connected to the computer 1 via USB cable 2. The electronic control unit 3 is powered by 24 Vdc via power cable 4. Each micropump driver 25 receives a digital signal generated by the microprocessor 23 and distributed by the distribution electronics board 24, converts the digital signal to a voltage control signal from 0 to 22 Vdc, and generates a 24 kHz ac signal having a peak-to-peak (p-p) amplitude equal or proportional to the input voltage of the voltage control signal. The ac signals from the drivers 25 are sent to the micropump unit 7 via multi-wire cable 5.

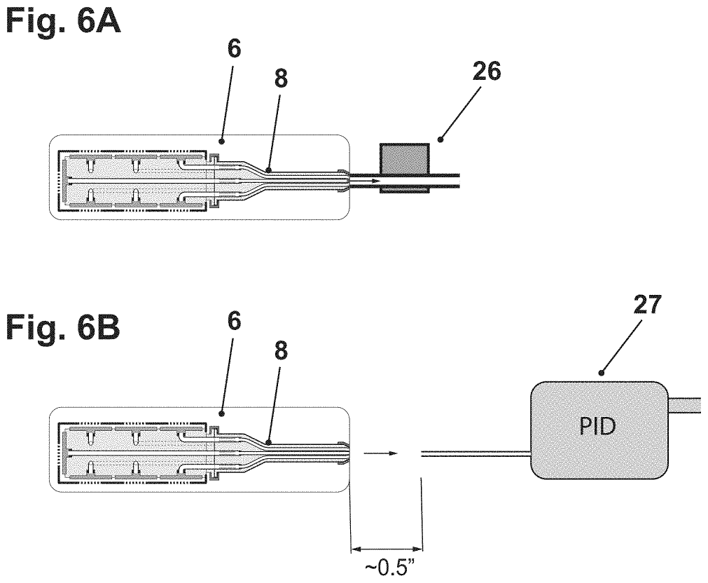

The odor delivery system of FIG. 1 can generate controllable flows of odorants from the odor delivery device 6. The software control implemented by the computer 1 allows both instantaneous and gradual switching of the micropumps 12 on and off, which creates increases and decreases of odor concentrations just underneath the user's nose. In one embodiment, each micropump 12 is capable of producing an air flow up to about 60 mL/min, which is sufficient to evoke a natural odor sensation but without the user being disturbed by the airflow and without the neighbors perceiving the odors, even of an offensive nature. As shown in FIG. 6A, the airflow can be measured using an electronic flow meter 26 directly attached to the outlet of the odor delivery device 6. As shown in FIG. 6B, the concentration of the odorant emitted from the device can be measured by a photo-ionization device (PID) 27 (e.g., a miniPID from Aurora Scientific of Ontario, Canada).

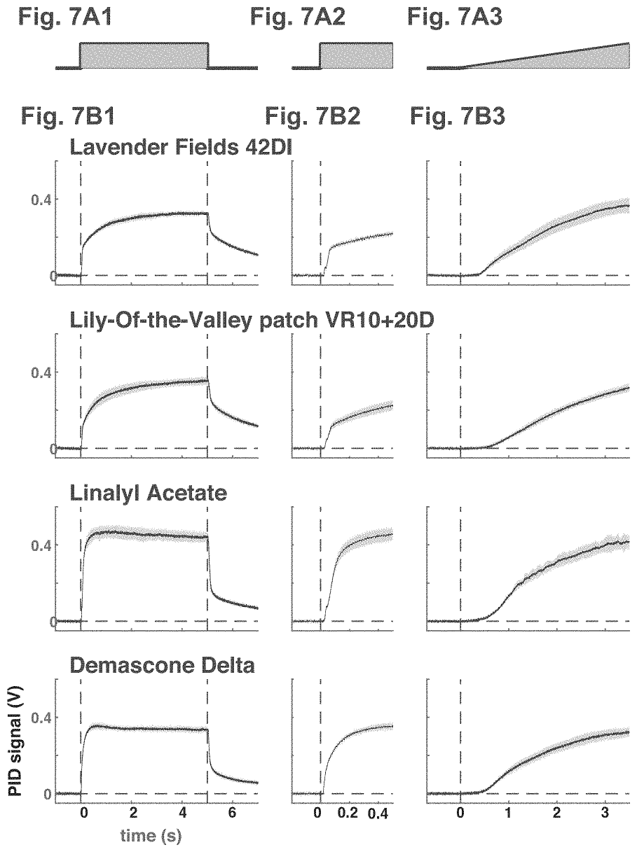

A rectangular temporal control pulse of 5-sec duration (as shown in FIG. 7A1) sent to the one of the micropump drivers 25 of FIG. 5, produces a measurable odor response. The temporal dependences of concentration of the emitted odorants measured by a photoionization detector is shown in FIG. 7B1 for two single molecular odorants (Linalyl Acetate and Demascone Delta) and two complex scents (Lavender Fields 42D1 and Lilly-of-the-Valley patch VR10+20D). FIG. 7A2 and FIG. 7B2 shows the same dependences for an expanded time scale. The concentration of the odorants reaches almost maximum level in the first 200 ms from the onset of the control pulse. This shows that the device can deliver odorant at time intervals much faster than human inhalation.

The micropumps can produce a slow ramp as shown in FIG. 7A3. Such a ramp produces a slow raise of the odorant concentration as shown in FIG. 7B3.

The temporal profiles of the odor concentration pulses are very reproducible. Solid black lines on FIGS. 7B1-7B3 show an average response for 10 individual trials. The shadow gray areas show standard deviation across 10 trials. The differences between individual trials are very small (±6%) and perceptually undetectable.

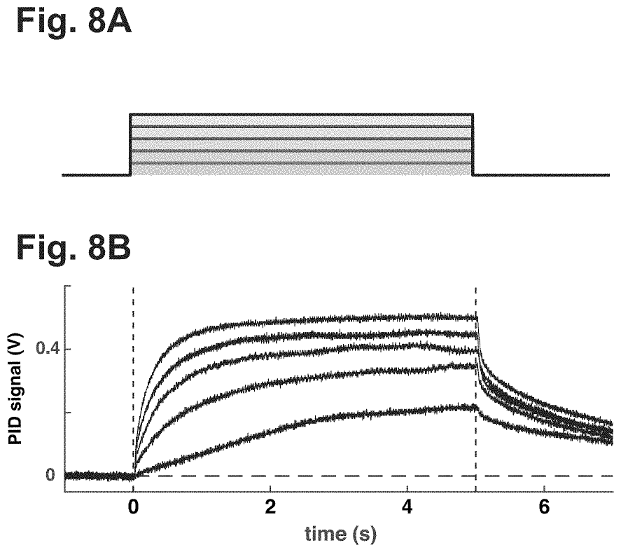

The concentration of the delivered odorant can also be controlled by changing an amplitude of the control signal. In one embodiment, the maximum airflow measured by the electronic flow meter 26 is 60 mL/min. Lowering of the airflow below a maximum value produces a proportionally less intense odor sensation. The minimal controllable odorant concentration is 10 times less than the maximal concentration. As shown in FIGS. 8A-8B, increasing the control pulse amplitude (FIG. 8A) from 4V to 8V, 12V, 16V, and 20V, produces stronger airflow and generates odor pulses with increasing concentrations (FIG. 8B). An expert can calibrate a desired concentration effect for a user by manipulating the temporal amplitude profile of a control pulse.

The maximal odorant concentration can be adjusted using different levels of dilution of the pure odorant during impregnation of the odorant carrier material 21 prior to the loading of the cartridge 7.

In one embodiment, to increase the number of odor presentations for a single cartridge, the cartridge is filed with a higher odorant concentration, which requires lower air flow to evoke a desired concentration effect. As an odorant material is used in the cartridge, the later odor deliveries are produced with increased airflow, thus compensating for the dilution of the odorant in the cartridge.

The micropumps 12 can be switched on individually or simultaneously. Simultaneous activation of two or more odorant channels allows for creating odorant mixtures with controllable ratios.

The speed with which a micropump 12 can be switched on and then off is faster than the duration of a human inhalation. This enables control of odorant concentration in sub-sniff temporal resolution. In addition, slow up and down concentration ramps may create novel odor perceptual effects, and can be used to control a user's adaptation to an odor and/or to create odor puffs or bursts effects.

Each compartment of the odor cartridge 8 can be loaded with a non-liquid material 21 soaked in one or several fragrance oils. The generation of fragrance compositions using multiple fragrance ingredients is described in U.S. Patent Publication Nos. 20020066798, 20020068009, 20020068010, 20050147523, and 20060196100, the teachings of all of which are incorporated herein by reference in their entirety. Materials able to be soaked are well described in the literature such as silica, EVA polymer, PE polymer, blotting paper, mineral or organic material, and other suitable solid, waxy, or gel-like materials as long as the material does not obstruct the air path.

In certain embodiments, an odor delivery device is capable of delivering odors within 0 to 4 inches from a nostril, without disturbing breathing patterns and breathing airflow and with a strength below what another subject's nose would smell 10-15 inches away.

In certain embodiments, a system of micropumps does not requiring a blank line and allows a flow dosage per odor and between odors impacting the odor delivery but not the natural breathing air flow of a person.

In certain embodiment, the device allows for a control of overall odor intensity across all channels by an individual user by scaling odor intensity of individual channels. Such a procedure will preserve the temporal sequence of the odor delivery steps, while adjusting overall intensity for an individual user, similar to a control of overall loudness of a music track.

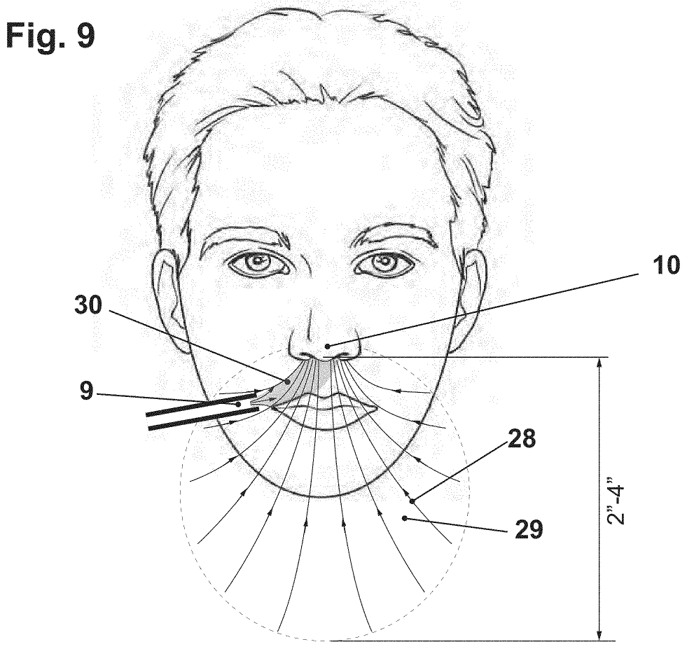

The device capitalizes on the physics of the inhalation and exhalation process (see airflow lines 28 of FIG. 9). A resting human, on average, inhales approximately 0.5 liters at each inhalation, and thus most of the air in the vicinity of the human nose inside this volume is inhaled into the nose. An approximate radius of a sphere 29 of volume V=500 mL is

$$L = {( \frac{3}{4 π} V )}^{1 / 3} \approx 5 cm \approx 2 inches .$$

Thus, if a device nozzle can be positioned at a distance from the user's nose of −2 inches, virtually all of the odorized air 30 emerging from the nozzle 9 will be then be diverted to the user's nose 10. The air flow from the nozzle is approximately 60 mL/min or 1 ml/sec, which is on average approximately 200 times lower than the air flow during inhalation. This ensures that all or nearly all of the odorized air from the device will be captured by a user's nose, as shown on FIG. 9. The precise geometry of the inhaled volume may depend on multiple complex factors, such as a shape of a user's nose, air flow in the room, and an actual inhalation volume and the rate of inhalation, but the vast difference between inhalation and flow rates and the small volume of odor delivery compensates for any of these factors.

Positioning the device's outlet closer to the nostril than the limit described above ensures effective delivery of the odorized air to the user's nose.

In between inhalations odorized air emerging from the device dissipates very quickly in the larger volume around the nose. The concentration of the odorized air will decrease in inverse proportion to the squared distance from the nozzle. At a distance exceeding 10-15 inches, the amount of odorized air present will be at concentrations that are undetectable by another subject.

There exist a broad range of concentrations, which are fully perceived by a user during inhalation and not detected by another nearby subject at a distance 10-15 inches. This range of concentration allows significant modulation of odor intensities, to create multiple odor perceptual effects.

### Alternative Embodiments

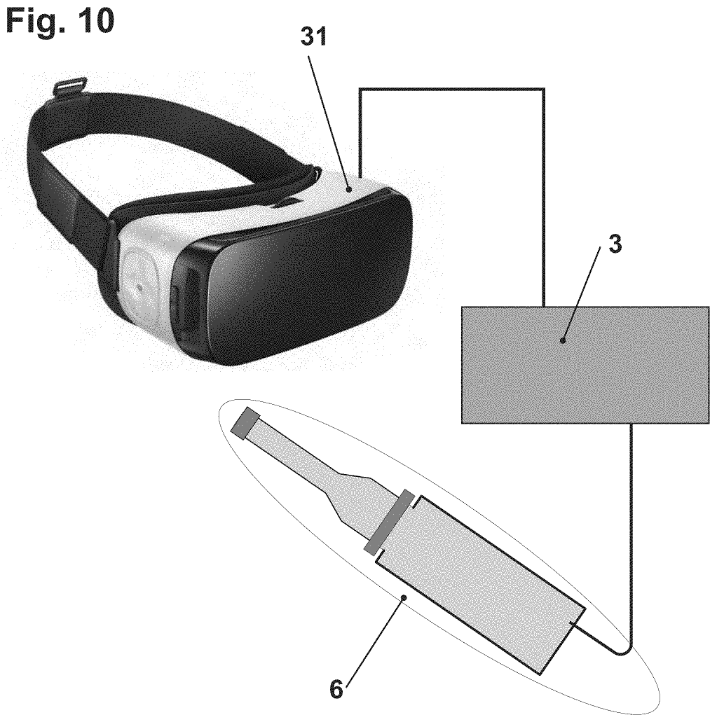

As shown in FIG. 10, in a different embodiment, computer 1 can be replaced by a virtual reality device 31 such as, for example, a VR headset from Oculus VR of Menlo Park, Calif.

In a different embodiment, the electronic control unit 3 can be embedded into the odor delivery device 6.

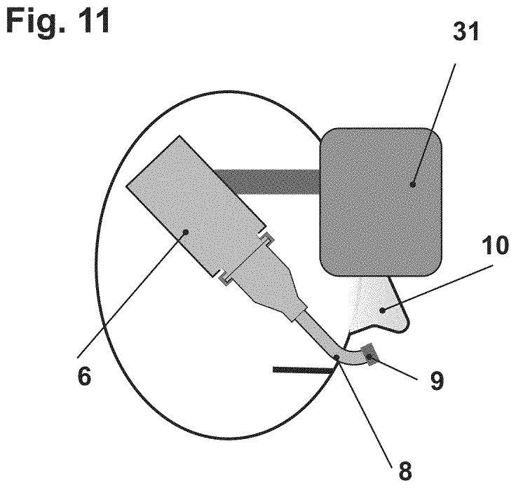

In a different embodiment shown on FIG. 11, the odor delivery device 6 can be mounted on the user's head or on the VR device 31. The device 6 can be powered by a battery (not shown) or from the power source from the other virtual reality device 31. In such a wearable embodiment, the tubing of the odor cartridge 8 is shaped in a way to fit user's head, and the position of the nose piece 9 can be adjusted to be placed near the user's nose 10.

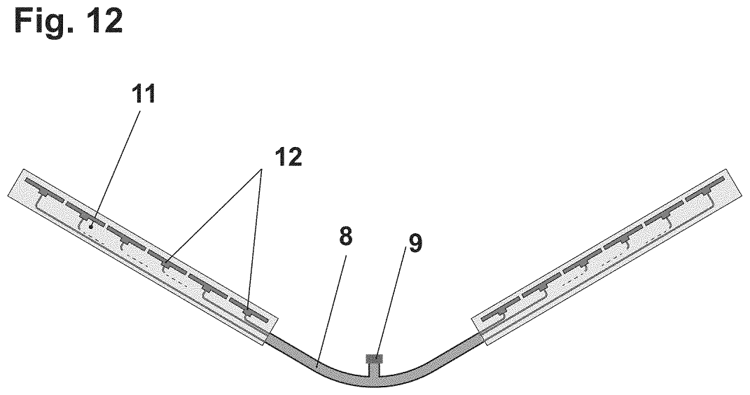

In different embodiments, the odor delivery device 6 may contain a number of odorant channels and a number of micropumps 12 greater or less than seven. In addition or alternatively, the micropumps 12 and connecting tubing 13 may be positioned differently from that shown in FIG. 2. The micropump unit 7 may have one, two, or more blocks positioned on the user's head. For example, two sets of micropumps 12 and odor cartridges 8 can be positioned on the left and right sides of the user's head and have one nose piece 9, as represented in FIG. 12, or two nose pieces.

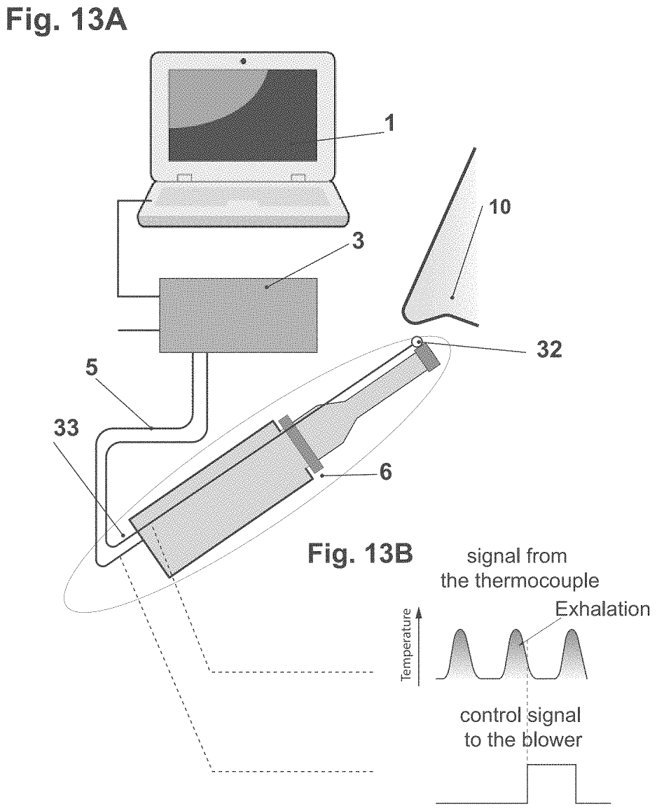

In a different embodiment shown at FIG. 13A, an odor delivery device 6 is equipped with a detector 32 for detecting and measuring the inhalations and exhalations of the user's respiratory pattern. The detector 32 can be a pressure, air velocity, and/or temperature measurement device, for example, a thermocouple or any other suitable, small detector for measuring respiration. Such addition allows for synchronization of odor delivery with the user's inhalation and/or exhalation. The signal from the detector 32 is transmitted to the electronic control unit 3 by the wires 33, or wirelessly. The activation of one or a few micropumps 12 is triggered by a specific phase of the respiratory cycle, for example, the end of exhalation, so that the odor delivery starts when the user inhales. See schematics at FIG. 13B.

In a different embodiment, controlling the timing of odor delivery to a user's respiration cycle allows creation of very precise pulses of odor, which are released from the device only during a user's inhalation.

In such an embodiment, activation of the device only during inhalation allows for delivery of higher odor concentration to the user without contamination of the environment, due to the fact that during inhalation all odorized air is inhaled into the user's nose, and in between inhalations the odor delivery is switched off.

In certain embodiments, different odors delivered concurrently by an odor delivery device are mixed only outside of the device nozzle in the region adjacent the user's nostrils.

In certain embodiments, the invention is a wearable device in weight/volume and battery powered so that it could be worn comfortably on a human head.

Although embodiments have been described in the context of odor delivery devices having micropumps, in general, embodiments can be implemented using other types of air pumps for delivering odors, such as small fans or pressurized air lines with proportional valves, which will allow fast and gradual control of airflow.

Although embodiments have been described in the context of odor delivery devices having (i) a single set of tubing for each air pump and (ii) no valves, in alternative embodiments, each air pump may be associated with one or more sets of tubing. In such embodiments, one or more suitable valves may be used to control which set of tubing is connected to an air pump that is associated with two or more sets of tubing.

According to certain embodiments, an article of manufacture comprises an odor delivery device comprising a plurality of independently controllable air pumps and dedicated tubing for each air pump configured to receive air pumped by the corresponding air pump and dispense the pumped air from at output of the odor delivery device. For each of at least two of the tubings, the tubing is configurable to receive scented material such that, when air is pumped by the corresponding air pump through the tubing, scented air flow is dispensed by the tubing from the odor delivery device. Each tubing ends near or at the output of the odor delivery device. If two or more tubings that are configured with two or more differently scented materials end near the output of the odor delivery device, then corresponding differently scented air flows concurrently dispensed by the two or more tubings are mixed together within an interior volume at the end of the odor delivery device before the resultant mixed air flow is dispensed from the odor delivery device. If two or more tubings that are configured with two or more differently scented materials end at the output of the odor delivery device, then corresponding differently scented air flows concurrently dispensed by the two or more tubings are mixed together only outside the odor delivery device.

According to at least some of the above embodiments, the air pumps are micropumps.

According to at least some of the above embodiments, the odor delivery device comprises a pump unit comprising the air pumps and dedicated tubing for each air pump and a cartridge unit configured to be removably mated to the pump unit. The cartridge unit comprises dedicated tubing for each air pump in the pump unit. When the cartridge unit is mated to the pump unit, each tubing in the cartridge unit mates with corresponding tubing in the pump unit. At least two of the tubings in the cartridge unit are configurable to receive scented material.

According to at least some of the above embodiments, each tubing in the cartridge unit is configurable to receive scented material.

According to at least some of the above embodiments, each tubing in the cartridge unit is narrowed at the output of the odor delivery device in order to inhibit backflow of air into the tubing.

According to at least some of the above embodiments, the odor delivery device further comprises a nose piece configured to be removably mated to the cartridge unit at the output of the odor delivery device.

According to at least some of the above embodiments, each tubing in the cartridge unit extends to the output of the odor delivery device.

According to at least some of the above embodiments, the article further comprises a controller configured to independently control each of the air pumps.

According to at least some of the above embodiments, the controller is configured to control the air pumps such that the concentration of odorant in the scented air flow dispensed from the output of the odor delivery device is both (i) high enough to be detected by a first person whose nose is within 4 inches of the device output and at an angle between 0 degrees and 135 degrees of the central air flow direction (i.e., the direction of the arrow in FIG. 6A) and (ii) low enough not to be detected by a second person whose nose is more than 15 inches of the device output.

According to at least some of the above embodiments, the controller comprises an electronic control unit.

According to at least some of the above embodiments, the electronic control unit is external to the odor delivery device.

According to at least some of the above embodiments, the electronic control unit is internal to the odor delivery device.

According to at least some of the above embodiments, the controller is configured to control the air pumps to concurrently deliver two or more differently scented air flows from the odor delivery device.

According to at least some of the above embodiments, the controller is (i) configured to receive respiration information about a user of the odor delivery device and (ii) configurable to control the air pumps to deliver one or more scented air flows from the odor delivery device only during inhalation by the user.

According to at least some of the above embodiments, the controller is (i) configured to receive respiration information about a user of the odor delivery device and (ii) configurable to control the air pumps to increase and/or decrease the rate of air flow through one or more tubings during an inhalation by the user.

According to at least some of the above embodiments, at least one tubing is configured with no scented material, the controller is configurable to control the corresponding air pump to selectively dispense a clean air flow from the odor delivery device.

According to at least some of the above embodiments, the controller is configurable to control the air pumps as part of a virtual reality/augments reality (VR/AR) system that coordinates the control of the air pumps as part of operation of the VR/AR system.

According to at least some of the above embodiments, the controller is configurable to coordinate the control of the air pumps with motions of the user during the operation of the VR/AR system.

According to at least some of the above embodiments, each tubing has an internal diameter of ⅛ inch or less and an internal diameter of 1/16 inch or less at the output of the odor delivery device.

According to at least some of the above embodiments, the internal diameter of each tubing is about 1/16 inch and the internal diameter of each tubing is about 1/32 inch at the output of the odor delivery device.

According to at least some of the above embodiments, the odor delivery device has no valves and no rotating mechanisms.

According to at least some of the above embodiments, each tubing is made of a polymer.

Embodiments of the invention may be implemented using (analog, digital, or a hybrid of both analog and digital) circuit based-processes, including possible implementation as a single integrated circuit (such as an ASIC or an FPGA), a multi-chip module, a single card, or a multi-card circuit pack. As would be apparent to one skilled in the art, various functions of circuit elements may also be implemented as processing blocks in a software program. Such software may be employed in, for example, a digital signal processor, micro-controller, general purpose-computer, or other processor.

Also for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements.

The functions of the various elements shown in the figures, including any functional blocks labeled as “processors,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non-volatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

It should be appreciated by those of ordinary skill in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

Unless explicitly stated otherwise, each numerical value and range should be interpreted as being approximate as if the word “about” or “approximately” preceded the value or range.

It will be further understood that various changes in the details, materials, and arrangements of the parts which have been described and illustrated in order to explain embodiments of this invention may be made by those skilled in the art without departing from embodiments of the invention encompassed by the following claims.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

All documents mentioned herein are hereby incorporated by reference in their entirety or alternatively to provide the disclosure for which they were specifically relied upon.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

It should be appreciated by those of ordinary skill in the art that any block diagrams herein represent conceptual views of illustrative circuitry embodying the principles of the disclosure. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

What is claimed is:

1. An article of manufacture comprising an odor delivery device, the odor delivery device comprising:
a plurality of independently controllable air pumps; and
dedicated tubing for each air pump configured to receive air pumped by the corresponding air pump and dispense the pumped air from an output of the odor delivery device, wherein:
for each of at least two of the tubings, the tubing is configurable to receive scented material such that, when air is pumped by the corresponding air pump through the tubing, scented air flow is dispensed by the tubing from the odor delivery device;
each tubing ends near or at the output of the odor delivery device;
if two or more tubings that are configured with two or more differently scented materials end near the output of the odor delivery device, then corresponding differently scented air flows concurrently dispensed by the two or more tubings are mixed together within an interior volume at the end of the odor delivery device before the resultant mixed air flow is dispensed from the odor delivery device; and
if two or more tubings that are configured with two or more differently scented materials end at the output of the odor delivery device, then corresponding differently scented air flows concurrently dispensed by the two or more tubings are mixed together only outside the odor delivery device.

2. The article of claim 1, wherein the air pumps are micropumps.

3. The article of claim 1, wherein the odor delivery device comprises:
a pump unit comprising the air pumps and dedicated tubing for each air pump; and
a cartridge unit configured to be removably mated to the pump unit, wherein:
the cartridge unit comprises dedicated tubing for each air pump in the pump unit;
when the cartridge unit is mated to the pump unit, each tubing in the cartridge unit mates with corresponding tubing in the pump unit; and
at least two of the tubings in the cartridge unit are configurable to receive scented material.

4. The article of claim 3, wherein each tubing in the cartridge unit is configurable to receive scented material.

5. The article of claim 3, wherein each tubing in the cartridge unit is narrowed at the output of the odor delivery device in order to inhibit backflow of air into the tubing.

6. The article of claim 3, wherein the odor delivery device further comprises a nose piece configured to be removably mated to the cartridge unit at the output of the odor delivery device.

7. The article of claim 3, wherein each tubing in the cartridge unit extends to the output of the odor delivery device.

8. The article of claim 1, further comprising a controller configured to independently control each of the air pumps.

9. The article of claim 8, wherein the controller is configured to control the air pumps such that the concentration of odorant in the scented air flow dispensed from the output of the odor delivery device is both (i) high enough to be detected by a first person whose nose is within 4 inches of the device output and at an angle between 0 degrees and 135 degrees of the central air flow direction and (ii) low enough not to be detected by a second person whose nose is more than 15 inches of the device output.

10. The article of claim 8, wherein the controller comprises an electronic control unit.

11. The article of claim 10, wherein the electronic control unit is external to the odor delivery device.

12. The article of claim 10, wherein the electronic control unit is internal to the odor delivery device.

13. The article of claim 8, wherein the controller is configured to control the air pumps to concurrently deliver two or more differently scented air flows from the odor delivery device.

14. The article of claim 8, wherein the controller is (i) configured to receive respiration information about a user of the odor delivery device and (ii) configurable to control the air pumps to deliver one or more scented air flows from the odor delivery device only during inhalation by the user.

15. The article of claim 8, wherein the controller is (i) configured to receive respiration information about a user of the odor delivery device and (ii) configurable to control the air pumps to increase and/or decrease the rate of air flow through one or more tubings during an inhalation by the user.

16. The article of claim 8, wherein, when at least one tubing is configured with no scented material, the controller is configurable to control the corresponding air pump to selectively dispense a clean air flow from the odor delivery device.

17. The article of claim 8, wherein the controller is configurable to control the air pumps as part of a virtual reality/augments reality (VR/AR) system that coordinates the control of the air pumps as part of operation of the VR/AR system.

18. The article of claim 17, wherein the controller is configurable to coordinate the control of the air pumps with motions of the user during the operation of the VR/AR system.

19. The article of claim 1, wherein each tubing has an internal diameter of ⅛ inch or less and an internal diameter of 1/16 inch or less at the output of the odor delivery device.

20. The article of claim 19, wherein the internal diameter of each tubing is about 1/16 inch and the internal diameter of each tubing is about 1/32 inch at the output of the odor delivery device.

21. The article of claim 1, wherein the odor delivery device has no valves and no rotating mechanisms.

22. The article of claim 1, wherein each tubing is made of a polymer.

23. The article of claim 1, wherein:
the air pumps are micropumps;
the odor delivery device comprises:
a pump unit comprising the micropumps and dedicated tubing for each micropump;
a cartridge unit configured to be removably mated to the pump unit; and
a nose piece configured to be removably mated to the cartridge unit at the output of the odor delivery device, wherein:
the cartridge unit comprises dedicated tubing for each micropump in the pump unit;
when the cartridge unit is mated to the pump unit, each tubing in the cartridge unit mates with corresponding tubing in the pump unit;
each tubing in the cartridge unit is configurable to receive scented material;
each tubing in the cartridge unit is narrowed at the output of the odor delivery device in order to inhibit backflow of air into the tubing; and
each tubing in the cartridge unit extends to the output of the odor delivery device;
each tubing has an internal diameter of ⅛ inch or less and an internal diameter of 1/16 inch or less at the output of the odor delivery device;
the odor delivery device has no valves and no rotating mechanisms; and
each tubing is made of a polymer.

24. The article of claim 23, further comprising an electronic control unit configured to independently control each of the micropumps, wherein:
the electronic control unit is configured to control the micropumps such that the concentration of odorant in the scented air flow dispensed from the output of the odor delivery device is both (i) high enough to be detected by a first person whose nose is within 4 inches of the device output and (ii) low enough not to be detected by a second person whose nose is more than 15 inches of the device output;
the electronic control unit is configured to control the micropumps to concurrently deliver two or more differently scented air flows from the odor delivery device;
the electronic control unit is configurable to control the micropumps to deliver one or more scented air flows from the odor delivery device only during inhalation by the user;
the electronic control unit is configurable to control the micropumps to increase and/or decrease the rate of air flow through one or more tubings during an inhalation by the user;
when at least one tubing is configured with no scented material, the electronic control unit is configurable to control the corresponding micropump to selectively dispense a clean air flow from the odor delivery device;
the electronic control unit is configurable to control the micropumps as part of a VR/AR system that coordinates the control of the micropumps as part of operation of the VR/AR system; and
the electronic control unit is configurable to coordinate the control of the micropumps with motions of the user during the operation of the VR/AR system.
